(19) United States
(12) Patent Application Publication
(10) Pub. No.: US 2018/0169427 A1
(43) Pub. Date: Jun. 21, 2018

(54) ATMOSPHERIC-PRESSURE PLASMA APPARATUS

(71) Applicant: PLABIO CO., LTD., Seoul (KR)

(72) Inventor: Buil Jeon, Gyeonggi-do (KR)

(21) Appl. No.: 15/900,072

(22) Filed: Feb. 20, 2018

Related U.S. Application Data
(63) Continuation of application No. PCT/KR2016/006829, filed on Jun. 27, 2016.

(30) Foreign Application Priority Data
Sep. 23, 2015 (KR) 10-2015-0134377

(51) Int. Cl. A61N 1/44

(52) U.S. Cl. CPC A61N 1/44; A61B 18/042

(57) ABSTRACT

An atmospheric-pressure plasma apparatus for skin care includes a roller electrode rotating in contact with a skin and having a surface coated with a dielectric material. A rotation shaft support part is electrically connected to the roller electrode and supports a rotation shaft of the roller electrode. A power supply is provided for applying an AC high voltage to the rotation shaft support part to generate a plasma between the roller electrode and the skin. A case part is provided for mounting a portion of the roller electrode while protruding to the outside.

### CROSS-REFERENCE TO RELATED APPLICATIONS

This application is a continuation of and claims priority to PCT/KR2016/006829 filed on Jun. 27, 2016, which claims priority to Korea Patent Application No. 10-2015-0134377 filed on Sep. 23, 2015, the entireties of which are both incorporated by reference herein.

### TECHNICAL FIELD

The present disclosure relates to atmospheric-pressure plasma apparatuses and, more particularly, an atmospheric-pressure plasma apparatus for skin care which employs a roller electrode.

### BACKGROUND

In general, a person's skin includes an epidermal layer, a dermal layer, and a subcutaneous fat. To manage the skin, nutrients should be steadily fed to the skin and wastes and toxins stacked on the skin should be removed. A method of feeding the nutrient includes a method of allowing cosmetics or vitamins to penetrate the skin, and a method of removing the nutrients or toxins includes make-up, massage or fomentation.

Among matters, a solid is a matter having the lowest energy state. A solid slows turns to a liquid and then transitions to a gas. When sufficient energy of ionization energy or more is applied to a neutral gas, plasma consisting of electrons, ions, neutral atoms, and molecules may be generated by ionization and electron-ion bond.

In plasma, nearly the same amount of positive charges and negative charges are mixed. Since the positive charges and the negative charges are maintained in an electrically neutral state while performing Brownian motion like free particles, the plasma is called ionized conductive gaseous species.

Among various plasma classification criteria such as plasma density, electron temperature, the degree of thermal equilibrium between species, generation manner, and applications, the plasma density and the electron temperature are used most basically. According to the degree of thermal equilibrium, plasma may be classified into local thermal equilibrium (LTE) and non-LTE. The term “local thermal equilibrium (LTE)” refers to a thermodynamic state where the temperatures of all of the plasma species are the same in the localized areas in the plasma.

Atmospheric-pressure plasma is utilized in fields of application such as surface modification and coating of a material and purification of environment. In recent years, studies on the atmospheric-pressure plasma have extended to applicability of biological applications and biomedical fields. An atmospheric-pressure plasma jet apparatus has been intensively studied in the biomedical field.

An electrode structure of a plasma jet is mostly a needle electrode structure, and various configuration manners are being studied according to power supply. In general, a plasma is generated by externally injecting an inert gas and applying a high voltage to the needle electrode structure. There has been introduced a dielectric barrier discharge (DBD) type plasma jet apparatus in which a small configuration is mounted on the center of a small-sized circular parallel plate.

Many types of power supplies for a plasma jet have been introduced according to driving frequency. A power supply of low-frequency discharge of tens to hundreds of kilohertz (KHz) includes a DC pulse power supply and an AC power supply. A MHz to GHz range of high-frequency discharge includes a radio-frequency (RF) and microwave (MW) generator. Power consumption of each power supply varies depending on fields of application. Power consumed by a plasma jet for use in biomedical fields is mostly 100 watts or less.

A plasma jet is mainly used for disinfection and sterilization. Various studies have been made on applications such as dental bleaching, destruction of pathogens, and blood coagulation. Important issues in the biomedical applications are compactness of a high-voltage electrode, easiness in controlling the high-voltage electrode, and safety security of a human body.

To directly apply a high-voltage electrode to cells of a living body or a human body, it is important to secure stability against an electric impact. Accordingly, the high-voltage electrode should not come in direct contact with a sample and a human body to secure the stability. A driving voltage applied to the electrode needs to decrease, and the amount of plasma current needs to be controlled within a small value of 1 to 2 mA to prevent an electric impact from applying to the living body. In 60 Hz commercial frequency AC which is minimum current that a human body can sense, an adult male can sense about 1 mA and sensed current increases as a frequency increases. The adult male feels pain at a current of 7 to 8 mA, and current higher than 7 to 8 mA is electrically and thermally dangerous.

An apparatus for providing skin stimulation using plasma has been studied. In Korean Patent Registration No. 10-1212749, “an apparatus for treatment of dermatological conditions” is disclosed. In this invention, discharge between a power electrode and a ground electrode is performed using the power electrode and the ground electrode to expose plasma to a skin. As the skin approaches the plasma, the skin is exposed to the plasma. Although this structure makes stable plasma discharge possible, it is difficult to perform plasma treatment according to a skin shape. Moreover, since many electrical reactions may occur at one point of the skin, the skin may suffer an electrical burn. In the structure using the power electrode and the ground electrode, a plasma is generated before the skin comes in contact with the structure. Therefore, power consumption increases.

A technique for stimulating skin along a shape of the skin is required. In addition, a technique for preventing arc discharge from occurring between a ground electrode and a power electrode is required.

### SUMMARY

Example embodiments of the present disclosure provide an atmospheric-pressure plasma apparatus for providing a stable plasma beauty treatment while minimizing skin friction and providing a comfortable feeling.

An atmospheric-pressure plasma apparatus according to an example embodiment of the present disclosure includes: a roller electrode rotating in contact with a skin and having a surface coated with a dielectric material; a rotation shaft support part electrically connected to the roller electrode and supporting a rotation shaft of the roller electrode; a power supply applying an AC high voltage to the rotation shaft support part to generate a plasma between the roller electrode and the skin; and a case part mounting the roller electrode, wherein a portion of the roller electrode protrudes to the outside.

In an example embodiment, the roller electrode may be in the form of a cylinder and has a through-hole penetrating the center of the roller electrode.

In an example embodiment, the rotation shaft support part may be inserted into the through-hole of the roller electrode.

In an example embodiment, the roller electrode may be in the form of a cylinder and may include a cylindrical auxiliary rotation shaft extending to protrude from opposite ends of the roller electrode in a central axis direction of the roller electrode.

In an example embodiment, the rotation shaft support part may support the auxiliary rotation shaft of the roller electrode.

In an example embodiment, the case part may further include a ground member coming in contact with a person's hand.

In an example embodiment, the roller electrode may be in the form of a cylinder and may have a plurality of trenches formed on a circumferential surface of the cylinder in a central axis direction of the cylinder.

In an example embodiment, the trench may include a region having first width and a region having second width.

In an example embodiment, the roller electrode may be in the form of a cylinder and may have a plurality of ring-shaped trenches formed on a circumferential surface of the cylinder in an azimuthal direction of the cylinder.

In an example embodiment, the trench may include a region having first width and a region having second width.

In an example embodiment, the roller electrode may be in the form of a cylinder and may have a plurality of holes formed on a circumferential surface of the cylinder.

In an example embodiment, the power supply may include: a battery disposed inside the case part; an AC generator receiving DC power from the battery to generate and supply AC power to the roller electrode; and a controller controlling the AC generator.

### BRIEF DESCRIPTION OF THE DRAWINGS

The present disclosure will become more apparent in view of the attached, example drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the present disclosure.

FIG. 1 is a perspective view of an atmospheric-pressure plasma apparatus according to an example embodiment of the present disclosure.

FIG. 2 is a cross-sectional view illustrating a roller electrode and a skin of the atmospheric-pressure plasma apparatus in FIG. 1.

FIG. 3 is a conceptual diagram of the atmospheric-pressure plasma apparatus in FIG. 1.

FIG. 4 illustrates an atmospheric-pressure plasma apparatus according to another example embodiment of the present disclosure.

FIG. 5A is a perspective view of a roller electrode of an atmospheric-pressure plasma apparatus according to another example embodiment of the present disclosure.

FIG. 5B is a cross-sectional view of the roller electrode in FIG. 5B.

FIG. 6A is a perspective view of a roller electrode of an atmospheric-pressure plasma apparatus according to another example embodiment of the present disclosure.

FIG. 6B is a cross-sectional view of the roller electrode in FIG. 6A.

FIG. 7A is a perspective view of a roller electrode of an atmospheric-pressure plasma apparatus according to another example embodiment of the present disclosure.

FIG. 7B is a cross-sectional view of the roller electrode in FIG. 7A.

FIG. 8 is a front view illustrating a pattern of a roller electrode according to another example embodiment of the present disclosure.

FIG. 9 is a front view illustrating a pattern of a roller electrode according to another example embodiment of the present disclosure.

### DETAILED DESCRIPTION

An atmospheric-pressure plasma apparatus according to an example embodiment of the present disclosure may provide skin stimulation using plasma. The atmospheric-pressure plasma apparatus may mount a battery together with an electric shaver therein, and the battery may be recharged from an external power supply. Additionally, the atmospheric-pressure plasma apparatus includes a case part that has a shape similar to a shaver to be held by a person's hand, a ground member is mounted on the case part, and the person's hand is electrically connected to the ground member. Thus, an electrical field is established between a roller electrode applied with a high voltage of the atmospheric-pressure plasma apparatus and a human body's skin desired to be treated. The electric field may generate atmospheric-pressure plasma through dielectric barrier discharge to stimulate and sterilize the skin.

The atmospheric-pressure plasma apparatus employs a rotatable roller electrode such that a friction with a skin may be minimized to provide a comfortable feeling caused by a contact. The shape of the roller electrode may be variously modified depending on a part of a human body. A surface of the roller electrode is coated with an insulator to suppress generation of an arc caused by a high voltage and an electric shock of the human body. The insulator may be made of a ceramic material such as aluminum oxide to be prevented from being readily removed.

The atmospheric-pressure plasma apparatus may improve convenience on the user's point of view as compared to a flat electrode and provide reduction in manufacturing cost.

The roller electrode is removable in the form of a sphere or cylinder, and many parts of the body may be managed by replacing the roller electrode.

Both a rotating portion and a supporting portion of the roller electrode may be made of a conductor and may naturally provide an electrical contact while rotating with an inserted structure with a slight allowance without a special mechanical structure. At a contact portion between the rotating portion and the supporting portion, they come in direct contact with each other to achieve an electrical contact. An outer portion of the other parts may be coated with an insulator or covered with an insulator to secure safety. The coated insulator may be a ceramic material such as alumina, Teflon or polyimide. Preferably, a thickness of the coated insulator may be between 50 and 500 micrometers. The thickness of the coated insulator may be decided considering insulation breakdown according to a dielectric constant and a voltage.

A plasma is generated when a distance between the skin and the roller electrode satisfies a predetermined condition. Thus, a pattern of various shapes may be formed on a surface of the roller electrode to constantly maintain the distance between the skin and the roller electrode. The pattern shape may include shapes of trench, hole, and protrusion that progress in a specific direction. Thus, while the roller moves along a skin, a plasma may be generated between the roller electrode and the skin and stimulate and sterilize the skin to provide a skin care effect.

Example embodiments will now be described more fully with reference to the accompanying drawings, in which some example embodiments are shown. Example embodiments may, however, be embodied in many different forms and should not be construed as being limited to the embodiments set forth herein; rather, these example embodiments are provided so that this disclosure will be thorough and complete, and will fully convey the scope of example embodiments of inventive concepts to those of ordinary skill in the art. In the drawings, the thicknesses of layers and regions are exaggerated for clarity. Like reference characters and/or numerals in the drawings denote like elements, and thus their description may be omitted.

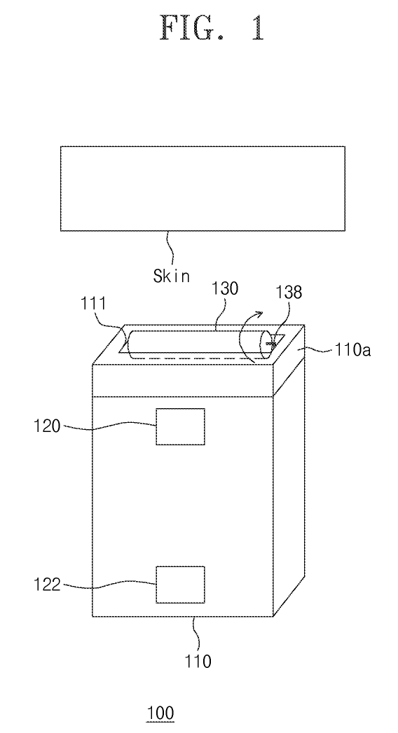

FIG. 1 is a perspective view of an atmospheric-pressure plasma apparatus according to an example embodiment of the present disclosure.

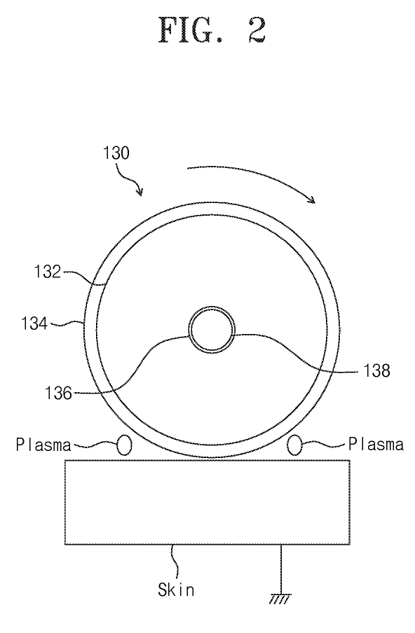

FIG. 2 is a cross-sectional view illustrating a roller electrode and a skin of the atmospheric-pressure plasma apparatus in FIG. 1.

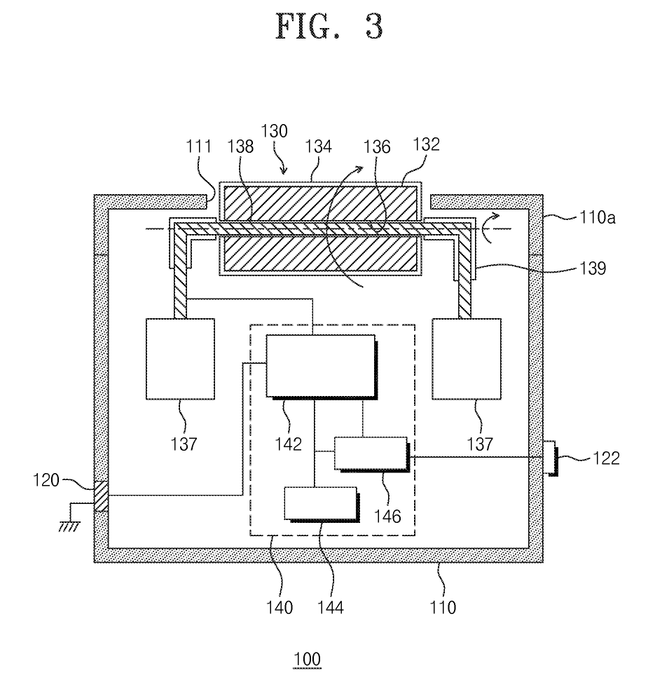

FIG. 3 is a conceptual diagram of the atmospheric-pressure plasma apparatus in FIG. 1.

Referring to FIGS. 1 through 3, an atmospheric-pressure apparatus 100 includes a roller electrode 130 rotating in contact with a skin and having a surface coated with a dielectric material, a rotation shaft support part 138 electrically connected to the roller electrode 130 and adapted to support a rotation shaft of the roller electrode 130, a power supply 140 applying an AC high voltage to the rotation shaft support part 138 to generate a plasma between the roller electrode 130 and the skin, and a case part 110 mounting the roller electrode 130. A portion of the roller electrode protrudes to the outside.

The roller electrode 130 may be in the form of a cylinder and have a through-hole 136 penetrating the center of the roller electrode 130. A body 132 of the roller electrode 130 may be made of aluminum, copper, a meal or a metal alloy. The roller electrode 130 may be coated with a dielectric material, and a thickness of a coating layer 134 may be in the range between tens and hundreds of micrometers. The coating layer 134 may block a direct contact between the skin and the roller electrode 130 to prevent current from flowing to a human body and provide an operation of dielectric barrier discharge. A material of the coating layer 134 may include at least one of alumina, Teflon, polyimide, a thermosetting resin, and an ultraviolet curing resin.

The rotation shaft support part 138 may provide the rotational degree of freedom to the roller 130 while being inserted into the through-hole 136 of the roller electrode 130 to be in electrical contact with the roller electrode 130. The roller electrode 130 and the rotation shaft support part 138 may be combined with each other with an allowance of several tens of micrometers without a special mechanical part.

The rotation shaft support part 138 may be a rod-shaped conductor having fixed rigidity. Opposite ends of the rotation shaft support part 138 may be curved to be combined with fixing parts 137, respectively. The fixing part 137 may be disassembled with or assembled with the rotation shaft support part 138. The fixing part 137 may be fixed to the case part 110. Except for a portion required to be electrically connected, the rotation shaft support part 138 may be coated with an insulator. A support coating layer 139 may include at least one of alumina, Teflon, polyimide, a thermosetting resin, and an ultraviolet curing resin. Alternatively, the rotation shaft support part 138 may be fabricated in the form of an insulatable case. Thus, unnecessary discharge between the human body and the rotation shaft support part 138 may be suppressed.

According to a modified embodiment of the present disclosure, a pair of bearings may be inserted into the through-hole 136 of the roller electrode 130 and the rotation shaft support part 138 may be combined with the bearing to stably rotate the roller electrode 130. In this case, the rotation shaft support part 138 and the bearing are electrically connected to each other.

The case part 110 may be made of an insulator of a plastic material. The case part 110 may have an opening to expose a portion of the roller electrode 130 to the outside. A periphery of the opening 111 may be disassembled from or assembled with the case 110. Thus, when a cover 110a is removed, the roller electrode 130 may be exposed and the roller electrode 130 may be disassembled to be replaced with another type of roller electrode or to be cleared.

The roller electrode 130 may be replaced removably from a case to use electrodes of various sizes and shapes according to parts of a human body and to provide convenient maintenance and management.

The case part 110 includes a ground portion 120. The ground portion 120 is contacted by a hand and electrically ground the human body. Thus, the power supply 140 supplies a high voltage between the roller electrode 130 and the skin.

The case part 110 may include a power switch 122. The power switch 122 may turn on/off an operation of the power supply 140.

The power supply 140 may include a battery 144 disposed inside the case part 110, an AC generator 142 receiving DC power from the battery 144 to generate and supply AC power to the roller electrode 130, and a controller 146 controlling the AC generator 142.

The battery 144 may be a rechargeable lithium-polymer battery or a disposable battery. The battery 144 may be disposed in an inner space of the case 110 and may provide the DC power to the AC generator 142.

The AC generator 142 may convert a DC voltage into an AC voltage. The AC generator 142 may include an inverter. A frequency of the AC voltage may be several kilohertz (KHz) to hundreds of KHz. A peak-to-peak AC voltage may be between 1 and 8 kV. A waveform of the AC generator 142 may be a sine wave. The AC generator 142 may output a high-voltage AC pulse as a pulse frequency of several Hz to several KHz. Specifically, the driving frequency of the AC generator 142 may be a 100 KHz region. The AC generator 142 may operate in a pulse mode of a pulse frequency of 10 to 1000 Hz. Stable dielectric barrier discharge may be performed at the pulse frequency and the driving frequency.

The controller 146 may adjust a duty ratio of a pulse of the AC generator 142 and control an output of the AC generator 146. In addition, the controller 146 may operate by the power switch 122.

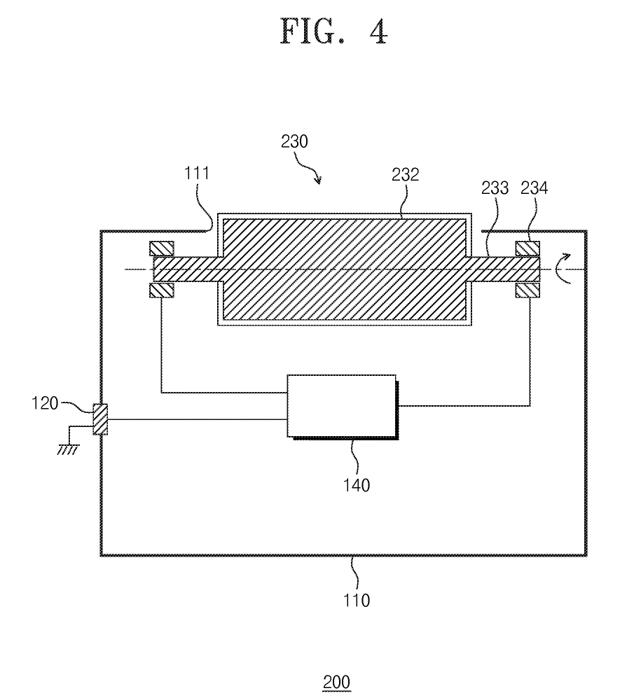

FIG. 4 illustrates an atmospheric-pressure plasma apparatus according to another example embodiment of the present disclosure.

Referring to FIG. 4, an atmospheric-pressure plasma apparatus 200 includes a roller electrode 230 rotating in contact with a skin and having a surface coated with a dielectric material, a rotation shaft support part 234 electrically connected to the roller electrode 230 and adapted to support a rotation shaft of the roller electrode 230, a power supply 140 applying an AC high voltage to the rotation shaft support part 234 to generate a plasma between the roller electrode 230 and the skin, and a case part 110 mounting a portion of the roller electrode 230 while protruding to the outside.

The roller electrode 230 may be in the form of a cylinder and include a cylindrical auxiliary rotation shaft 233 extending to protrude from opposite ends in a central axis direction. The roller electrode 230 may include a coating layer 232, and the coating layer 232 may cover an exposed surface of the roller electrode 230. A thickness of the coating layer 232 may be in the range from tens to hundreds of micrometers. The coating layer 232 may block a direct contact between the skin and the roller electrode 230 to prevent current from flowing to a human body and provide an operation of dielectric barrier discharge. A material of the coating layer 232 may include at least one of alumina, Teflon, polyimide, a thermosetting resin, and an ultraviolet curing resin.

The rotation shaft support part 234 may support the auxiliary rotation shaft 233 of the roller electrode 230. The rotation shaft support part 234 may have the same structure as a bearing and may be electrically connected to the auxiliary rotation shaft 233 while supporting the auxiliary rotation shaft 233.

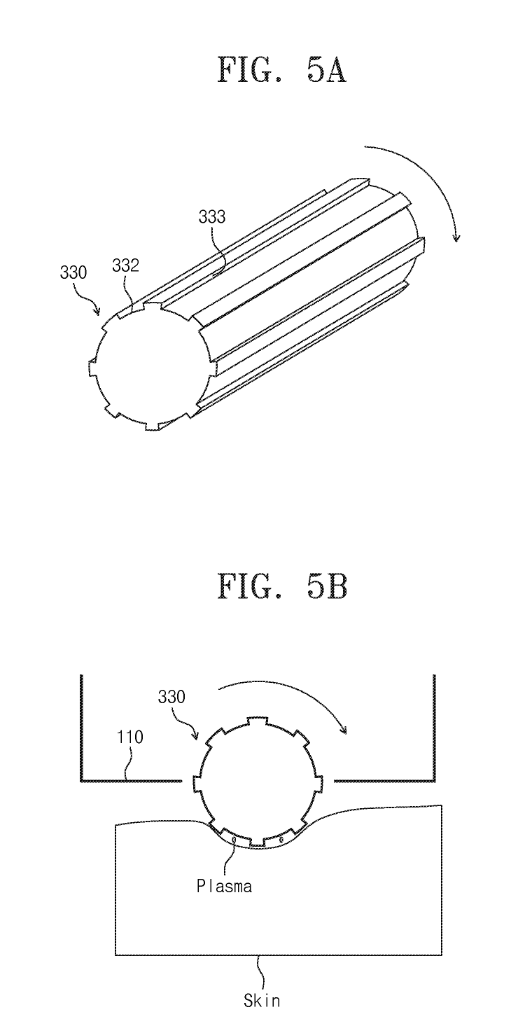

FIG. 5A is a perspective view of a roller electrode of an atmospheric-pressure plasma apparatus according to another example embodiment of the present disclosure.

FIG. 5B is a cross-sectional view of the roller electrode in FIG. 5B.

Referring to FIGS. 5A and 5B, a roller electrode 330 may be in the form of a cylinder and have a plurality of trenches 333 formed on a circumferential surface of the cylinder in a central axis direction of the cylinder.

Depth of the trench 333 is several millimeters or less and may be preferably in the range from 1 to 3 millimeters. A protruding portion of the roller electrode 330 may come in contact with a human body to prevent generation of a plasma, and a plasma may be generated in an empty space between the inside of the trench 333 and the skin. A coating layer of the roller electrode 330 may cover a surface of the roller electrode 330.

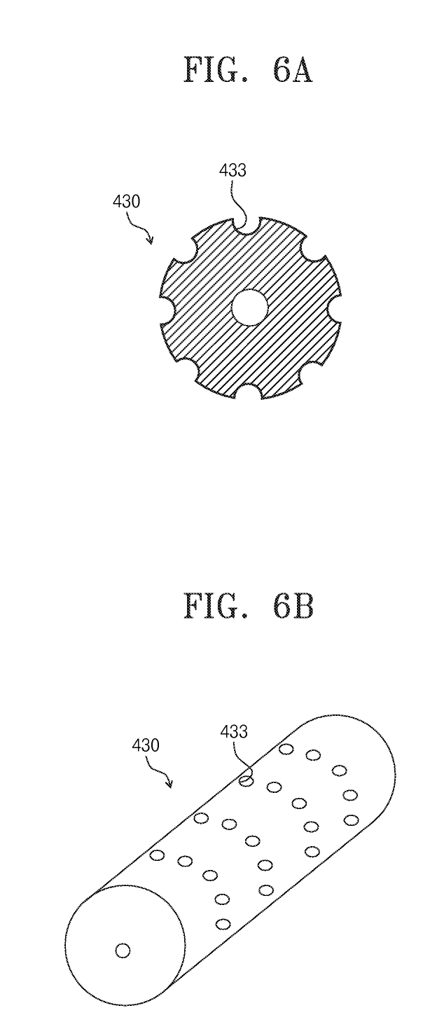

FIG. 6A is a perspective view of a roller electrode of an atmospheric-pressure plasma apparatus according to another example embodiment of the present disclosure.

FIG. 6B is a cross-sectional view of the roller electrode in FIG. 6A.

Referring to FIGS. 6A and 6B, a roller electrode 430 may be in the form of a cylinder and have a plurality of holes 433 formed on a circumferential surface of the cylinder. Depth of the hole 433 may be several millimeters or less and may be preferably in the range from 1 to 3 millimeters. The holes 433 may be distributed on a surface of the roller electrode 430 at reregulate intervals. A coating layer of the roller electrode 430 may cover the surface of the roller electrode 430.

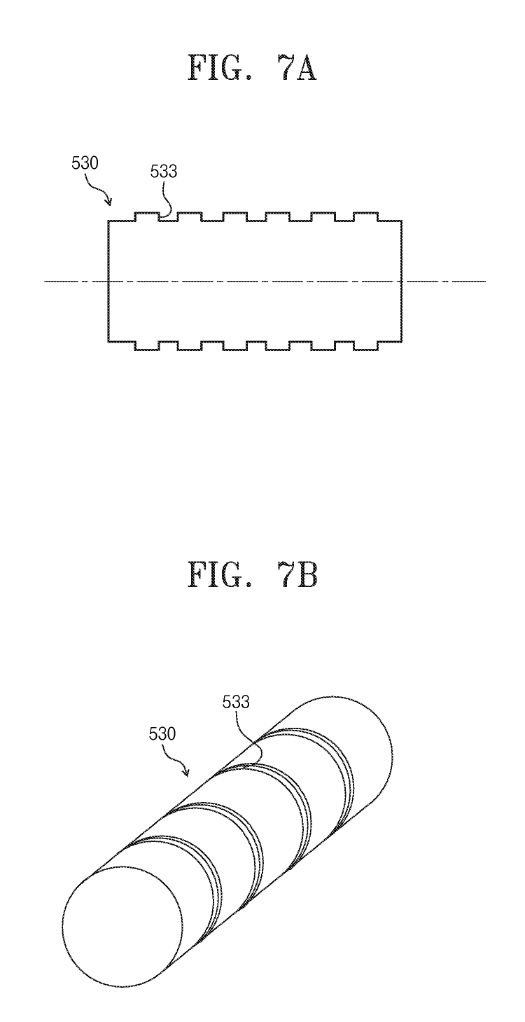

FIG. 7A is a perspective view of a roller electrode of an atmospheric-pressure plasma apparatus according to another example embodiment of the present disclosure.

FIG. 7B is a cross-sectional view of the roller electrode in FIG. 7A.

Referring to FIGS. 7A and 7B, a roller electrode 530 may be in the form of a cylinder and have a plurality of ring-shaped trenches 533 formed on a circumferential surface of the cylinder in an azimuthal direction of the cylinder. The trench 533 may be formed to cover the circumference of the roller electrode 530. Depth of the trench 533 may be several millimeters or less and may be preferably in the range from 1 to 3 millimeters. The trenches 533 may be arranged in a central axis direction of the roller electrode 530 at regular intervals. A coating layer of the roller electrode 530 may cover a surface of the roller electrode 530.

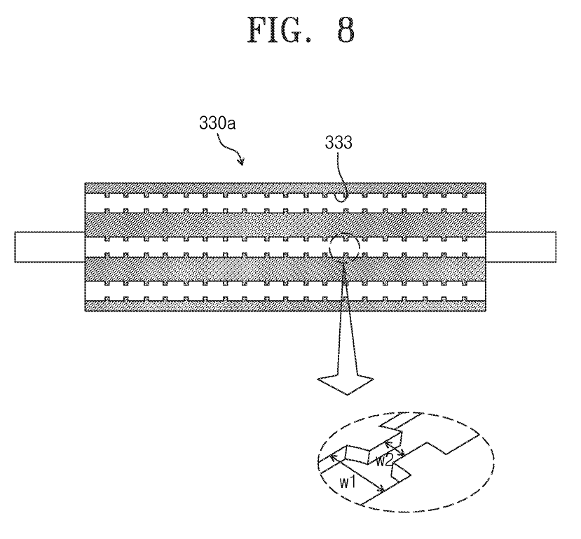

FIG. 8 is a front view illustrating a pattern of a roller electrode according to another example embodiment of the present disclosure.

Referring to FIG. 8, a roller electrode 330a may be in the form of a cylinder and have a plurality of trenches 333 formed on a circumferential surface of the cylinder in a central axis direction of the cylinder. The trench 333 may include a region having first width and a region having second width.

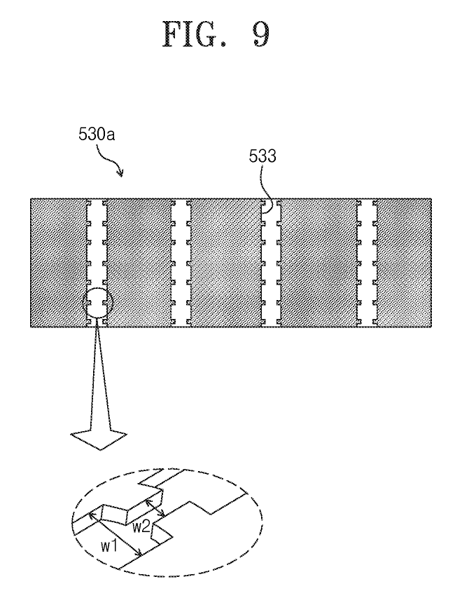

FIG. 9 is a front view illustrating a pattern of a roller electrode according to another example embodiment of the present disclosure.

Referring to FIG. 9, a roller electrode 530a may be in the form of a cylinder and have a plurality of trenches formed on a circumferential surface of the cylinder in a central axis direction of the cylinder. The trench 533 may include a region having first width and a region having second width.

According to example embodiments of the present disclosure, as a roller electrode rotates along a skin, the roller electrode may provide a comfortable feeling without creating a friction with the skin. In addition, patterns formed on a surface of the roller electrode may be maintained at stable discharge intervals and provide spatially uniform discharge.

According to example embodiments of the present disclosure, a roller electrode may be easily replaced in various forms, may be easy to carry, and may be removed to be easily cleared.

Although the present disclosure and its advantages have been described in detail, it should be understood that various changes, substitutions and alterations can be made herein without departing from the spirit and scope of the disclosure as defined by the following claims.

What is claimed is:

1. An atmospheric-pressure plasma apparatus comprising:
a roller electrode for rotating in contact with skin and having a surface coated with a dielectric material;
a rotation shaft support part electrically connected to the roller electrode and supporting a rotation shaft of the roller electrode;
a power supply for applying an AC high voltage to the rotation shaft support part to generate a plasma between the roller electrode and the skin; and
a case part mounting the roller electrode,
wherein a portion of the roller electrode protrudes outside the atmospheric-pressure plasma apparatus.

2. The atmospheric-pressure plasma apparatus of claim 2, wherein the roller electrode is in a form of a cylinder and has a through-hole penetrating a center of the roller electrode.

3. The atmospheric-pressure plasma apparatus of claim 2, wherein the rotation shaft support part is inserted into the through-hole of the roller electrode.

4. The atmospheric-pressure plasma apparatus of claim 1, wherein the roller electrode is in a form of a cylinder and includes a cylindrical auxiliary rotation shaft extending to protrude from opposite ends of the roller electrode in a central axis direction of the roller electrode.

5. The atmospheric-pressure plasma apparatus of claim 4, wherein the rotation shaft support part supports the auxiliary rotation shaft of the roller electrode.

6. The atmospheric-pressure plasma apparatus of claim 4, wherein the case part further includes a ground member coming in contact with a person's hand.

7. The atmospheric-pressure plasma apparatus of claim 1, wherein the roller electrode is in a form of a cylinder and has a plurality of trenches formed on a circumferential surface of the cylinder in a central axis direction of the cylinder.

8. The atmospheric-pressure plasma apparatus of claim 7, wherein the trench includes a region having first width and a region having second width.

9. The atmospheric-pressure plasma apparatus of claim 1, wherein the roller electrode is in a form of a cylinder and has a plurality of ring-shaped trenches formed on a circumferential surface of the cylinder in an azimuthal direction of the cylinder.

10. The atmospheric-pressure plasma apparatus of claim 1, wherein the trench includes a region having first width and a region having second width.

11. The atmospheric-pressure plasma apparatus of claim 1, wherein the roller electrode is in a form of a cylinder and has a plurality of holes formed on a circumferential surface of the cylinder.

12. The atmospheric-pressure plasma apparatus of claim 1, wherein the power supply comprises:
a battery disposed inside the case part;
an AC generator receiving DC power from the battery to generate and supply AC power to the roller electrode; and
a controller controlling the AC generator.
